(19) United States
(12) Patent Application Publication
(10) Pub. No.: US 2004/0236216 A1
(43) Pub. Date: Nov. 25, 2004

(54) System and method for simulating imaging data

(76) Inventors: Ravindra Mohan Manjeshwar, Guilderland, NY (US); Melvin Kurt Simmons, Schenectady, NY (US); Michael Christopher Montalto, Albany, NY (US); Eric Dustin Agdeppa, Latham, NY (US)

Correspondence Address: Patrick S. Yoder, FLETCHER YODER, P.O. Box 692289, Houston, TX, 77269-2289, US

(21) Appl. No.: 10/881,182

(22) Filed: Jun. 30, 2004

Related U.S. Application Data
(63) Continuation-in-part of application No. 10/413,828, filed on Apr. 15, 2003.

(51) Int. Cl. A61B 6/00

(52) U.S. Cl. 600/436

(57) ABSTRACT

In accordance with one aspect of the present technique, an imaging simulator system comprises a processor assembly that includes a time activity module configured to generate a time activity data, an imager module adapted to receive at least one imager parameter and configured to model the acquisition of an imaging system, and a simulator module adapted to receive at least the time activity data, and the imager model and configured to generate simulated sensed data based on the time activity data, and the imager model.

### CROSS REFERENCE TO RELATED APPLICATIONS

This application is a continuation-in-part of U.S. patent application Ser. No. 10/413,828, entitled “Model Based Image Quality Optimization For Antibody PET Imaging Protocols,” filed Apr. 15, 2003, which is incorporated by reference.

### BACKGROUND

The invention relates generally to the field of nuclear imaging and, more specifically, to the generation and use of simulated image data.

In modern healthcare facilities, medical diagnostic and imaging systems are invaluable for identifying, diagnosing, and treating a variety of physical conditions including cancer, neuro-degenerative disorders, and cardiovascular disorders. Diagnostic and imaging systems which may be used in this manner include nuclear medicine and imaging techniques that use a radioactive tracer that targets a specific target area or tissue that may be of interest. The radioactive tracer is injected into a patient and the signal generated from the decay of the radioactive tracer is used to construct an image of the distribution of the radioactive tracer within the patient. Examples of nuclear imaging techniques that utilize radioactive tracers for imaging purposes include positron emission tomography (PET) systems, single positron emission computed tomography (SPECT) systems, multiple emission tomography (MET) systems, planar gamma camera imaging systems, and some imaging protocols for magnetic resonance imaging (MRI) systems.

For example, PET imaging techniques rely on the use of radioactive tracers that, upon decay, emit particles known as positrons. Upon emission, the positrons typically only travel a short distance before colliding with electrons, which carry an opposite charge. When a positron collides with an electron, the two particles annihilate one another and, in the process, generate two gamma rays that travel in opposite directions from one another. These gamma rays may be detected by a detector ring disposed about the patient. By detecting a number of annihilation events in this manner, an image may be generated which indicates the location and/or concentration of the tracer within the patient. If desired, a series of such images may be generated which indicate the location and concentration of the tracer over time.

There are numerous factors that affect the level of accuracy of an image, such as a PET image or other images generated using nuclear imaging techniques. These factors include selection of imaging components, their configuration, and their placement. For a given examination, it may therefore be desirable to select factors or examination conditions that will provide the desired image quality. Accordingly, various tools have been developed for simulating aspects of nuclear imaging so that one or more factors may be selected or configured based upon the results of the model. Typically, however, the models employed do not fully or satisfactorily address the biological and/or pharmacological aspects of the imaging process. For example, the physiological and/or structural aspects of the disease state may affect the pharmacokinetics of the radioactive tracer. Similarly, different organs and tissues may differentially process or absorb the radioactive tracer, even in non-diseased tissue. Because of these types of factors, the complex organ and tissue geometry that exists in vivo and the pharmacokinetic properties of the radioactive tracer over time may not be completely or accurately modeled by separate or discrete models. Thus, the existing simulation models may not be completely accurate for modeling a disease condition or the generation of images based on the biological and pharmacological properties of a radioactive tracer. For example, in the case of neuro-degenerative diseases such as Alzheimer's disease (AD), an imaging simulator model may not accurately reflect the accumulation of beta-amyloid (a principal indicator of Alzheimer's disease) in the brain or in regions of the brain. In such cases, the simulated data may not accurately reflect the biological and pharmacological factors related to the target brain tissue, the radioactive tracer, and the beta-amyloid.

Therefore, in order to optimize the quality of the final image obtained from the imaging system, there is a need for a simulation system model that models the biology behind the formation of a disease condition along with the pharmacokinetics of the radioactive tracer.

### BRIEF DESCRIPTION

In accordance with one aspect of the present technique, an imaging simulator system comprises a processor assembly that includes a time activity module configured to generate a time activity data, an imager module adapted to receive at least one imager parameter and configured to model the acquisition of an imaging system, and a simulator module adapted to receive at least the time activity data, and the imager model and configured to generate simulated sensed data based on the time activity data, and the imager model.

In accordance with another aspect of the present technique, a method of simulating a nuclear imaging process comprises the steps of generating a set of simulated sensed data based on at least a time activity data, and an imager model. Computer programs that afford functionality of the type defined by this method are also provided by the present technique.

In accordance with yet another aspect of the present technique, a method of selecting an imaging compound comprises the steps of specifying at least one of a time activity data, and an imager model to a simulator system, obtaining a set of simulation results from the simulation model wherein the set of simulation results are generated by the simulation system based on at least one of the time activity data and the imager model.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram illustrating an exemplary embodiment of a imaging simulator system;

FIG. 2 is a diagrammatical representation illustrating the interaction between various modules in accordance with embodiments of the present technique; and

FIG. 3 is a diagrammatical illustration of an embodiment of the imaging simulator system wherein the time activity module comprises a biological module that provides time activity data to the simulator module;

FIG. 4 is a diagrammatical illustration of an embodiment of the imaging simulator system wherein the time activity module comprises a phantom module that provides time activity data to the simulator module;

FIG. 5 is a diagrammatical illustration of an embodiment of the imaging simulator system wherein the time activity module comprises a pharmacokinetic module that provides time activity data to the simulator module;

FIG. 6 is a diagrammatical illustration of an embodiment of the imaging simulator system wherein the time activity module comprises a customizable time activity generator that provides time activity data to the simulator module;

FIG. 7 is a diagrammatical illustration of an embodiment of the imaging simulator system wherein the time activity data is generated by an interaction between a biological module and a pharmacokinetic module;

FIG. 8 is a diagrammatical illustration of an embodiment of the imaging simulator system wherein the time activity data is generated by an interaction between a pharmacokinetic module and a phantom module;

FIG. 9 is a diagrammatical illustration of an embodiment of the imaging simulator system wherein the time activity data is generated by an interaction between a biological module, a phantom module, and a pharmacokinetic module; and

FIG. 10 is a diagrammatical illustration of an exemplary method of using an imaging simulator system in accordance with embodiments of the present technique.

### DETAILED DESCRIPTION

The various aspects of the present technique disclosed below describe a model-based simulation system which may be used to evaluate radioactive tracers for use in a targeted imaging system and to optimize various imaging parameters to obtain better image quality. The present technique quantitatively models the physiological and physical pathways involved in the production of images. In this manner, this technique enables the comparison of effects of modulating imaging parameters and/or pharmacokinetics of the radioactive tracer on image quality in instances where the effects do not readily lend themselves to repeat experimental inquiry. From these comparisons, the effectiveness of new radioactive tracers or imaging protocols may be determined. Similarly, the interpretation of complex image data may be facilitated by using the present techniques to determine the physiological and physical parameters that might generate the image date.

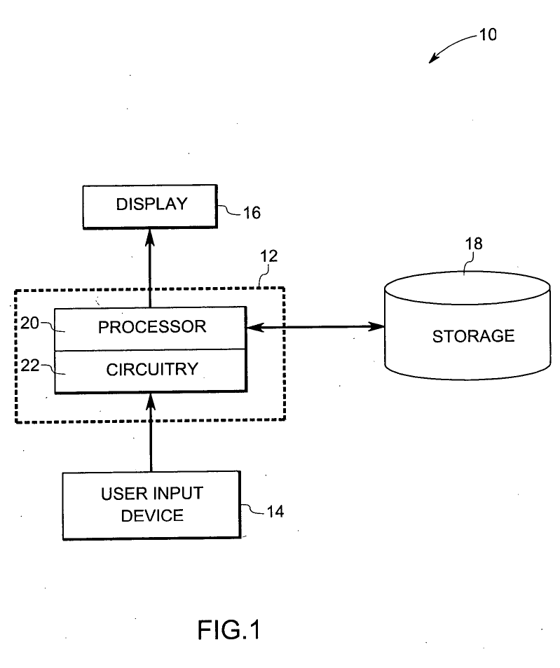

Turning now to the drawings and referring first to FIG. 1, an imaging simulator system 10 for use in accordance with the present technique is shown. The imaging simulator system 10, as depicted, includes a processor assembly 12, a user input device 14, a display device 16, and a storage device 18. The processor assembly 12 includes one or more processor 20 and circuitry 22. The processor 20 may include one or more processor. Examples of processors include parallel processors, networked processors, personal desktop assistants (PDAs), and other handheld microprocessor based devices. The circuitry 22 may include at least one of firmware, digital integrated circuits, analog integrated circuits, and mixed signal integrated circuits.

The processor assembly 12 integrates input data from various system and imaging models and generates simulated sensed data in accordance with the input data. The processor assembly 12 also processes the simulated sensed data in accordance with various imaging parameters to generate an image. The processor assembly 12 further analyzes the image to generate image adjusting parameters in the form of image quality metrics to optimize the image. The image simulator system 10 also includes a user input device 14 that allows a user to input data in the form of imaging parameters. The processor assembly 12 accesses storage 18 to store and retrieve data in the form of process parameters, and software for performing various operations as required. Examples of storage media that make the storage device 18 include removable hardware such as single hard drives, hard drives in a redundant array of independent disks (RAID) configuration, and compact disks such as CDROMs and DVDs. The imaging simulator system 10 also includes a display device 16 to display the image generated by the processor assembly 12. Examples of display devices may include cathode ray tube (CRT) based devices, and liquid crystal display (LCD) based devices.

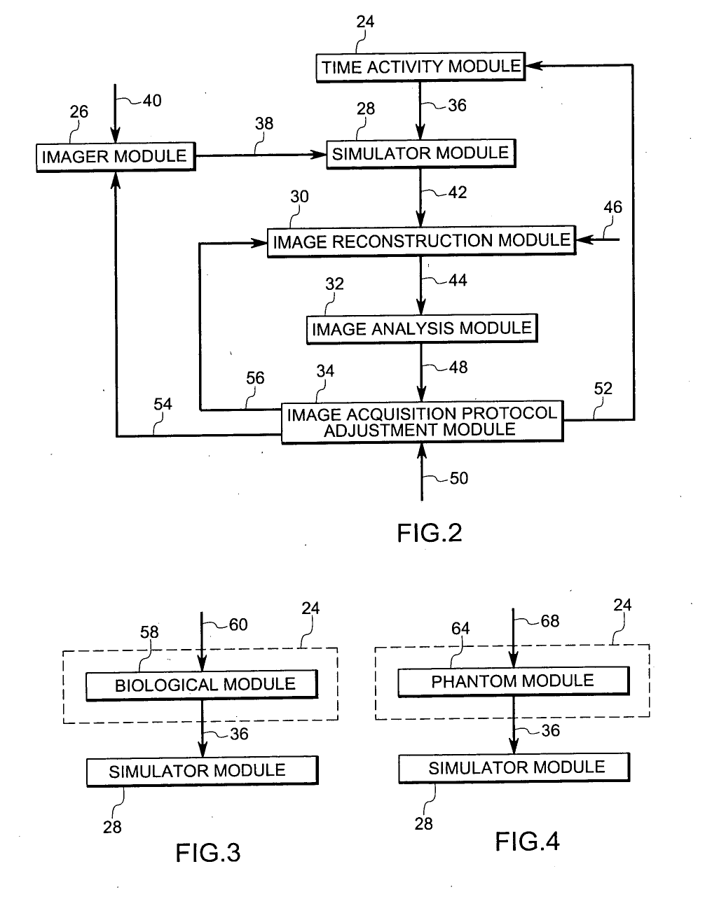

FIG. 2 depicts various modules and models that may be implemented by the imaging simulator system 10 depicted in FIG. 1. For example, as depicted in FIG. 2, the imaging simulator system 10 may implement a time activity module 24, an imager module 26, a simulator module 28, an image reconstruction module 30, an image analysis module 32, and an image acquisition protocol adjustment module 34. In addition, FIG. 2 also depicts possible interactions between the depicted modules in an exemplary implementation of the present technique.

In the depicted exemplary implementation, the time activity module 24 provides time activity data 36, either by generating time activity data through the operation of one or more algorithms or models or by providing pre-configured or user specified time activity data. The time activity data 36 may be provided as an input to the simulator module 28. The time activity data 36 may represent or account for a variety of input factors, such as the type of radioactive tracer to be modeled, and/or the decay factor and kinetics of the radioactive tracer. For example, in modeling time activity data directed toward amyloidogenesis and the study of Alzheimer's, the time activity data 36 may include information about the concentration of different amyloid species in relevant anatomic compartments, such as various brain regions, blood, and non-brain regions. Rates of production, clearance, oligomerization, and trafficking of the different amyloid species, as input or configured in the time activity module 24, may be factors in the generation of the time activity data 36. Various embodiments of the time activity module 24 are discussed in greater detail below.

In addition, it may be desirable to model the physics associated with an imaging modality or scanner, such as with an imager module 26, to generate synthetic or simulated images based on physiology-based pharmacokinetics of a radioactive tracer. In particular, the imager module 26 may be used to generate an imager model 38 to convert radioactive tracer concentrations in the physiologic and anatomic compartments into images based on a modeled imaging modality. The imager model 38 may be a simple analytical model, may be an extensive Monte-Carlo simulation model, or may model the desired imaging modality based on other quantitative or parametric principles.

The imager module 26 based on provided or configured imager parameters 40 may generate the imager model 38. The imager parameters 40 typically relate to a specific diagnostic medical imaging system to be modeled, such as a positron emission tomography (PET) system, a single photon emission computed tomography (SPECT) system, a magnetic resonance imaging (MRI) system, a multiple emission tomography (MET) system, a computed tomography (CT) system, an x-ray imaging system, an ultrasound imaging system, and an optical imaging system, such as a fluorescent optical imaging system. The imager parameters 40 applicable to each of these imaging systems may vary due to differences in selecting and using imaging compounds, capturing data, and processing data, as well as due to the different physical phenomena related to the respective imaging processes.

The imager parameters 40 may be provided as an input by a system operator based on the imaging system to be modeled or may be preconfigured or preset within the imager module 26. In general, the imager parameters 40 account for the physical processes associated with image acquisition, such as the attenuation of a signal by surrounding tissue, the geometric and intrinsic sensitivity and point spread function of the detectors, and so forth. For example, for a PET or SPECT imager, the imager parameters 40 may include information representing the shape and dimensions of a collimator, configuration of scintillator crystals, configuration of sensor arrays, as well as configuration settings for related processing circuitry. The output from the imager module 26, in the form of imager model 38, may be provided to the simulator module 28 for further processing.

The simulator module 28 uses the output from the time activity module 24 and the imager module 26 to compute simulated sensed data 42. Examples of simulated sensed data 42 may include a simulated sinogram. For example, in one embodiment of present technique, the simulator module 28 may integrate the outputs from the time activity module 24 based on the imager module 26 to generate simulated sensed data 42 representing image data which would be observed on an imaging modality described by the imager module 26 based on the time activity data 36.

The simulated sensed data 42 may be provided to an image reconstruction module 30 configured to construct an image 44 of the region of interest. The image reconstruction module 30 may reconstruct the simulated sensed data 42 based upon preset or preconfigured settings or based on one or more image reconstruction parameter 46 provided to the image reconstruction module 30. The reconstructed image 44 may be provided to an image analysis module 32 configured to generate image quality metrics 48. For example the generated image quality metrics 48 may include computational quantitative metrics, such as contrast-to-noise-ratio, or metrics like lesion detectability and binding potential. In addition to the image quality metrics 48, user feedback 50 may be provided to an image acquisition protocol adjustment module 34 to generate respective feedback signals 52, 54, and/or 56, which may alter the operation of one or more of the time activity module 24, the imager module 26 and/or the image reconstruction module 30, respectively.

While the preceding discussion illustrates various general aspects of the present technique, FIGS. 3-6 illustrate several exemplary embodiments that may be used to generate time activity data 36, as discussed above. For example, FIG. 3 illustrates one embodiment in accordance with aspects of present technique, wherein the time activity module 24 includes a biological module 58. The biological module 58 may accept as an input, biological parameters 60 pertaining to a target tissue or organ. The biological parameters 60 may include biochemical and/or biophysical parameters used to generate a biological model that contains time activity data 36, as depicted in FIG. 2. Biochemical parameters that may be employed include parameters that indicate chemical and biochemical activities in the human body and their changes over time. Examples of biochemical parameters include information about decay factors, blood composition, the interstitial fluid, brain lesions and so forth. Biophysical parameters of a substance that may be employed include parameters that indicate changes in physical and biophysical levels or state of the substance. Examples of biophysical parameters include the concentration, the clearance rate, and/or the rate of absorption of a substance in the region of interest, such as the radioactive tracer or a target of the radioactive tracer. The biological module 58 typically models the biology in a region of interest, including portions in the region of interest affected by a disorder, rather than relying on empirical data alone. The time activity data output from the biological module 58 in the form of a biological model may be provided into the simulator module 28 as the time activity data 36 of FIG. 2.

In another embodiment, in accordance with aspects of present technique, as illustrated in FIG. 4, the time activity module 24 may include a phantom module 64 to provide the simulator module 28 with a phantom model representative of time activity data 36, as illustrated in FIG. 2. A phantom is defined to be a digital representation of the geometry of the anatomical structure and the radioactive tracer distribution within that anatomical structure of the object being imaged. The phantom module 64 may be a library of digital phantom models readily accessible from a storage location by an imaging simulator system. A system operator may select one of the phantom models, a portion of the phantom model and/or alter a specific phantom model, such as via a configuration input 68, based on the desired time activity output. The output from the phantom module 64, in the form of a phantom model, may be provided to the simulator module 28.

In addition to the physiological activity in the imaged region, the pharmacokinetics of a radioactive tracer used may also be of interest. As noted above, the radioactive tracer contains a radioactive element selectively absorbed by a target tissue or organ having a disease condition or disorder. For example, a normally functioning tissue or organ absorbs the substance at a certain rate while a diseased or abnormal tissue or organ absorbs the radioactive element at a different, typically higher rate. Due to the presence of the radioactive element, the selective absorption by the tissue or organ with the disease condition can easily be quantified in terms of time and space kinetics within the physiologic and anatomic compartments of interest. Based on this quantification, pharmacokinetic parameters can be generated. A pharmacokinetic module 70, as shown in FIG. 5 may therefore be used to model these pharmacokinetic factors. For example, the pharmacokinetic module 70 may be provided with pharmacokinetic parameters 72 which are used to generate a pharmacokinetic model containing time activity data 36 that may be used in subsequent simulation steps. The pharmacokinetic parameters 72 may include factors that relate to the time activity of the radioactive tracer, including the type of radioactive tracer, decay factors, kinetics, affinity, compartmental volumes, clearance rates, transport rates, and biological half life. In general, the pharmacokinetic model of the radioactive tracer describes the time and space kinetics of the radioactive tracer within various physiologic and anatomic compartments to provide information about concentrations of the radioactive tracer over time. Examples of physiologic and anatomic compartments can include regions like blood vessels, the brain, and interstitial fluid. The output from the pharmacokinetic module 70, in the form of a pharmacokinetic model, may be provided to the simulator module 28 as time activity data 36.

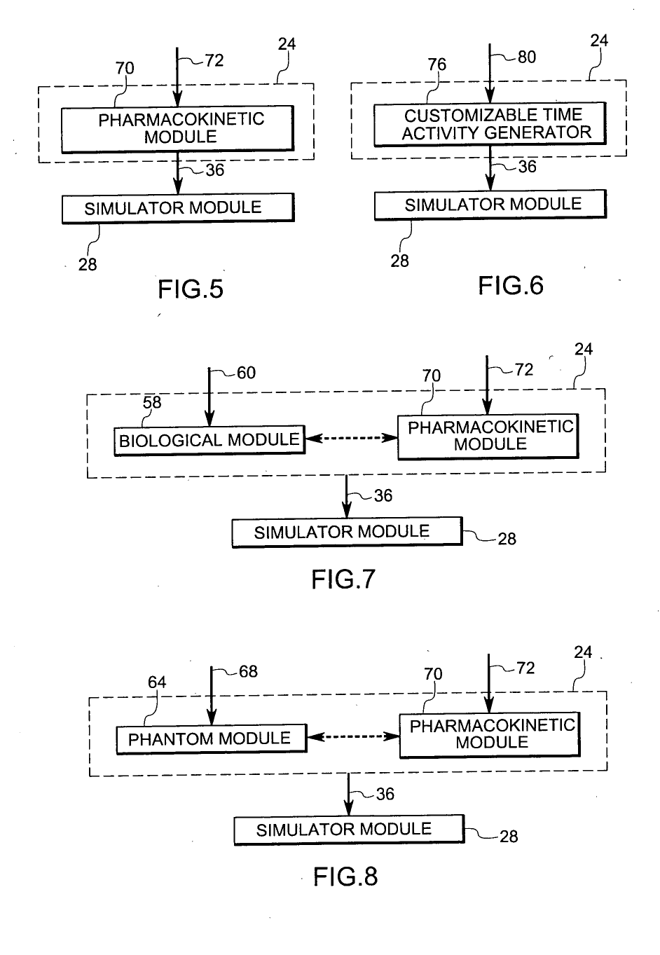

In yet another embodiment, in accordance with aspects of present technique, as illustrated in FIG. 6, the time activity module 24 may include a customizable time activity generator 76 that generates time activity curves, or other time activity descriptions, such as based on operator input 80. The time activity curves may represent or contain the time activity data 36 provided to the simulator module 28. The customizable time activity generator 76 may generate the time activity curves via operation of one or more routines or algorithms, including routines or algorithms adapted for implementation in an automated manner, such as on a general or special purpose computer.

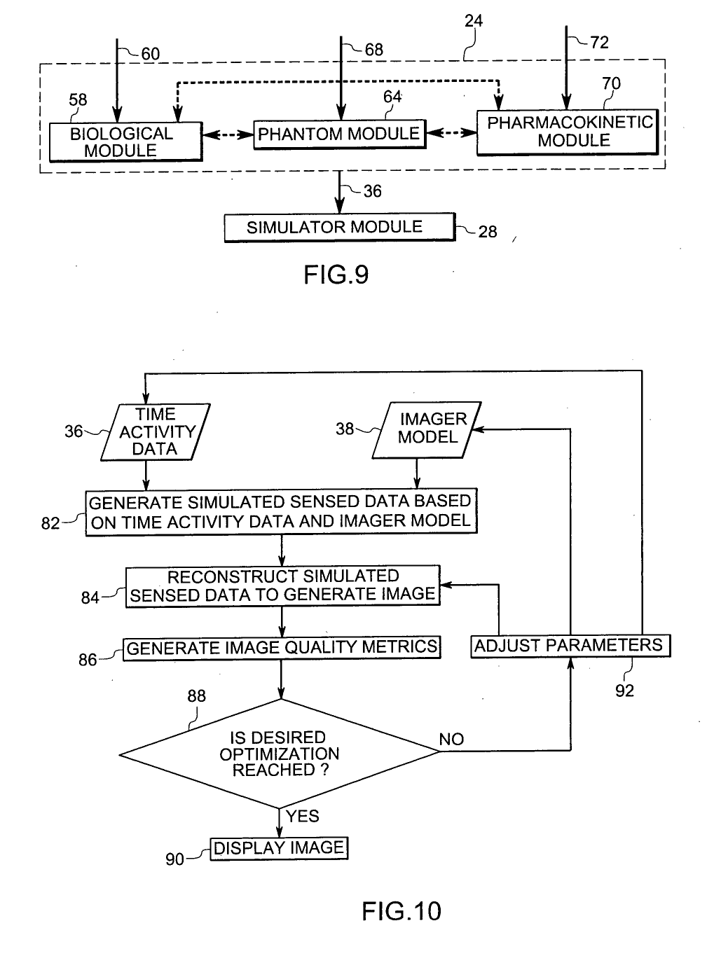

FIGS. 7-9 illustrate exemplary embodiments that depict interactions between various modules, as illustrated and described previously, that may constitute the time activity module 24. For example, FIG. 7 illustrates an exemplary embodiment wherein the time activity data 36 may be generated by an interaction between a biological module 58 and a pharmacokinetic module 70. Similarly, FIG. 8 illustrates an exemplary embodiment wherein the time activity data 36 may be generated by an interaction between a phantom module 64 and a pharmacokinetic module 70. FIG. 9 illustrates an exemplary embodiment wherein the time activity data 36 may be generated by an interaction between a biological module 58, a pharmacokinetic module 70 and a phantom module 64.

Referring now to FIG. 10, an exemplary method of using an imaging simulator system, as illustrated in FIG. 2 and described above, is depicted. In this embodiment, time activity data 36, and data from an imager model 38 may be used to generate simulated sensed data at step 82. The time activity data 36 may describe the time and space kinetics of the radioactive tracer within various physiologic and anatomic compartments, providing information about concentrations of the radioactive tracer in different compartments over time. For example, in one embodiment, the time activity data 36 may represent the combination of data generated by a biological module 58 and a pharmacokinetic module 70, as described above. The imager model 38 may include information about a specific imaging system to be modeled. At step 84, the simulated sensed data may be reconstructed to generate a simulated image for the specific imaging system. Based on the simulated image, image quality metrics may be generated at step 86. At step 88, the image quality metrics may be analyzed and compared with preconfigured or operator provided threshold levels. If the threshold level is exceeded, indicating that an optimization has been reached, the generated image may be displayed at step 90. If, however, the threshold level is not exceeded, indicating that an optimization has not been reached, adjustments to the time activity data 36, the imager model 38 and/or the image reconstruction parameters may be made at step 92 and reconstruction of the image using the adjusted simulated sensed data and/or an adjusted reconstruction process may be performed. The sequence of steps may be repeated until a desired optimization is reached.

Example: As described above, a technique for deriving simulated sensed data, which accounts for biological, pharmacokinetic and imager variables, is provided.

To illustrate the potential utility of these simulation techniques for tracer development and drug discovery, the effects of different tracer affinities on binding potential were simulated and synthetic images generated. Model parameters were set to reproduce levels of beta amyloid within a platelet-derived growth factor promoter expressing amyloid precursor protein (PDAPP) transgenic mouse. Pharmacokinetic curves of virtual tracers were computed and a Monte Carlo PET detector system was configured for a commercially available preclinical PET imaging system. In this way, the effects of beta amyloid therapy and tracer affinity were modeled and the ability to differentiate beta amyloid levels by PET imaging techniques were observed.

A biological model of beta amyloid was employed to represent the concentration of beta amyloid in different tissues and structures for which a simulated image was desired. The beta amyloid model described beta amyloid oligomerization and trafficking in the brain regions of interest. The equations representing the oligomerization process were replicated to create three independent sets of oligomerization equations, one for each brain region to be represented. The three regions used the same parameters for the elongation and fragmentation reactions. In this manner, the beta amyloid, i.e., biological, model was represented by the following set of differential equations, where j ranges over the regions of the brain

$$\frac{ⅆ {A}_{1 , j} ( t )}{ⅆ t} = {p}_{j} ( t ) + {r}_{PB} \frac{{V}_{j}}{{V}_{B}} P ( t ) + {r}_{CB} \frac{{V}_{j}}{{V}_{B}} C ( t ) - {r}_{BP} {A}_{1 , j} ( t ) - {r}_{BC} {A}_{1 , j} ( t ) - \\ {l}_{j} ( t ) {A}_{1 , j} ( t ) + f {\sum }_{i = 2}^{N} {A}_{i , j} ( t ) - e {A}_{1 , j} ( t ) ( 2 {A}_{1 , j} ( t ) + {\sum }_{i = 2}^{N} {A}_{i , j} ( t ) )$$

$$\frac{ⅆ {A}_{2 , j} ( t )}{ⅆ t} = e {A}_{1 , j}^{2} ( t ) + f {A}_{3 , j} ( t ) - e {A}_{1 , j} ( t ) {A}_{2 , j} ( t ) - \frac{1}{2} {fA}_{2 , j} ( t )$$

$$\frac{ⅆ {A}_{1 , j} ( t )}{ⅆ t} = e {A}_{1 , j} ( t ) {A}_{i - 1 , j} ( t ) + {fA}_{i + 1 , j} ( t ) - e {A}_{1 , j} ( t ) {A}_{i , j} ( t ) - {fA}_{i , j} ( t )$$

$$\frac{ⅆ P ( t )}{ⅆ t} = {p}_{P} ( t ) + {r}_{BP} {\sum }_{j} \frac{{V}_{j}}{{V}_{B}} {A}_{1 , j} ( t ) + {r}_{CP} C ( t ) - {r}_{PB} P ( t ) - {r}_{PC} P ( t ) - {l}_{P} ( t ) P ( t )$$

$$\frac{ⅆ C ( t )}{ⅆ t} = {p}_{C} ( t ) + {r}_{BC} {\sum }_{j} \frac{{V}_{j}}{{V}_{B}} {A}_{1 , j} ( t ) + {r}_{PC} P ( t ) - \\ {r}_{CB} C ( t ) - {r}_{CP} C ( t ) - {l}_{C} ( t ) C ( t )$$

where A_{i,j}(t) is the concentration of the beta amyloid oligomer of length i in region j of the brain, P(t) is the concentration of the beta amyloid monomer in the plasma, C(t) is the concentration of beta amyloid monomer in the cerebral spinal fluid (CSF), pⱼ(t) is the production rate of beta amyloid monomer in region j, and lⱼ(t) is the loss rate. The time dependence of pⱼ and lⱼ represents the different effects of therapies. The parameters r_{XY} represent the transport among the brain (_{B}), plasma (_{P}), and CSF (_{C}). Transport is modeled as a simple kinetic rate constant. The parameters e and f represent the addition and loss of monomer from an oligomer. Vⱼ is the volume of each region of the brain. N is an upper cut-off for numerical solutions of the differential equations. This value was set empirically to achieve less than 1% error from neglected higher-order terms. The value of N was between 24 and 32 for the runs used in both baseline and therapy conditions.

Only the monomer equation in each set (cortex, hippocampus, and cerebellum) includes rates for production and loss of beta amyloid monomer in the brain, and for transport to and from the plasma and CSF. The production rate was assumed to vary between regions, and was left as an adjustable parameter. The loss rate was kept equal in the three regions. To extend the transport model to regions of the brain, the overall rate constant was modified to make the volumes appear explicitly in the equations. Then the equations for the three regions were modified by adding data on the volumes of regions of the mouse brain, CSF, and plasma. The output of the model was a file with separate concentration predictions for each of the three brain regions for monomer and for all sizes of oligomer.

A pharmacokinetic model was applied to this output to derive concentration data in the different brain regions over time. The pharmacokinetic model was a physiologically based pharmacokinetic model of a perfusion-limited system. The pharmacokinetic model was written as a set of coupled differential equations based on parameters for volume and blood flow rates of mouse organs. The organs modeled were blood, liver, cortex, hippocampus, cerebellum, muscle, spleen, kidney, and lung. The only organ with a clearance rate was the liver, with a clearance rate of 10 ml/hr. The volumes of the brain regions were based on the beta amyloid mouse model described above. The total flow rate of plasma to the mouse brain was divided in proportion to the volume of each region. The perfusion terms in the model used a partition coefficient of 1 for all regions except liver, spleen, and kidney, which had partition coefficients of 2, and for the brain and CSF, which are discussed below.

Non-brain values were selected to simulate a kinetic response with a mean time in the plasma that matched measurements with [¹⁸F] MPPF. A simple diffusion-limited element was added for transfer between plasma and CSF, with a 1-hour time constant. This term had no significant effect on the time course of concentration in the plasma because there is so little target amyloid in the CSF. The decay of ¹⁸F was included in the equations with a half-life of 110 minutes. The pharmacokinetic model was implemented as differential equations. The injected activity of the tracer in the blood was set at t=0 to be 5×10⁻⁶ Ci/ml. The model was configured to generate time-activity curves for a period of 2 hours following injection. The partition coefficient of the tracer is determined by its binding to the predicted levels of beta amyloid within each region of the brain. We assumed a simple kinetic law for binding of the PET agent to beta amyloid. Two cases were explored: binding only to monomer, and equal binding to any oligomer. We explored a wide range of binding strengths, both stronger and weaker than the binding of beta amyloid oligomer to itself in the oligomerization model. In this kinetic model the partition coefficient for monomer in region j of the brain is

$${R}_{j} = 1 + \frac{1}{{k}_{d}} {A}_{1 , j} ( t )$$

while the partition coefficient for binding to all oligomers (including monomer) is

$${R}_{j} = 1 + \frac{1}{{k}_{d}} {\sum }_{i = 1}^{∞} {A}_{i , j} ( t )$$

The pharmacokinetic model was then run with the values of Rⱼ for the regions of the brain from the beta amyloid biological model described above, before and after simulated treatment, and with binding only to monomer or to oligomer molecules. This produced as output the time-activity curves needed as input to the PET system model.

In this example, a digital atlas from segmented high-resolution MRI images of a mouse brain was used to construct a digital phantom of 75 slices of 512×512 images with a voxel resolution of 0.02×0.02×0.25 mm³. The cortex, hippocampus, cerebellum and CSF compartments were assigned radiotracer activity concentrations from the pharmacokinetic curves generated by the tracer model. The mouse brain phantom was then used in further simulations. As noted herein, however, the time activity data itself, as opposed to a derived phantom, may be employed in the image simulation processes.

An imaging model describing the selected image system physics was also employed. A Monte-Carlo model of a small animal PET scanner was used to generate synthetic PET images of the mouse brain. The scanner model modeled the generation and transport of 511 keV photons through the mouse brain phantom, the detector ring and system geometry, gamma ray interactions within the detector module, image calibrations, corrections and reconstruction. The modeled scanner had four detector rings of 14.5 cm diameter. One detector ring consisted of 24 blocks of an 8×8 array of 2.1×2.1×10 mm mixed lutetium silicate (MLS) crystals. The modeled scanner had an absolute sensitivity of 2.4% and a resolution of ˜2.2 mm at the center of the field-of-view.

Based on this modeled scanner, imaging acquisition protocols were modeled. Simulated image acquisition was started 1,800 sec post-injection and the acquisition time was 3,600 sec. A fully three-dimensional acquisition was simulated with a 250-700 KeV energy window and 8 ns timing resolution. Multi-slice re-binning was used to generate two-dimensional sinograms. Sinograms were corrected for variations in detector crystal sensitivities, geometric radial repositioning and tissue attenuation. Images were reconstructed with the filtered back-projection algorithm with a Hanning window and calibrated to units of μCi/cc for quantization. Five replicate images were simulated for each time-activity curve. Regions-of-interest were manually drawn on the cortex, hippocampus and cerebellum. The mean and standard deviation of activity concentration were computed for quantitative accuracy in relation to the input time-activity curves to determine the percentage error in quantization.

The beta amyloid model was tuned by adjusting the production rates and transport rates to match within 5% of published concentrations of total beta amyloid (Aβ₄₀₊₄₂ summed over all length oligomers) in the three regions of the brain, and monomer concentrations in the plasma and the CSF. Total baseline beta amyloid concentrations were computed as cortex 4.9×10⁻⁷ M, hippocampus 1.1×10⁻⁶ M, cerebellum 1.61×10⁻⁷ M, plasma 3.41×10⁻¹¹ M, and CSF 3.36×10⁻⁹ M. The affinity of the tracer for Aβ was set at 30×10⁻⁹ M. The pharmacokinetic profile of a virtual tracer specific for beta amyloid peptide in the PDAPP transgenic mouse was computed and PET acquisitions were simulated at different time points to show how acquisition time can affect imaging output.

To illustrate how the present technique relates the pharmacokinetic parameters to imaging output, we varied the affinity of the virtual tracer and performed additional PET simulations. To demonstrate self-consistency in the scanner model, time-activity concentrations were calculated using only the output image data for each affinity. The calculated values approximated the input time-activity values, however there was a general trend for calculated values to be slightly lower, perhaps due to partial volume effects of the simulated scanner.

Binding potentials were generated using time-activity concentrations computed from the image output for the hippocampus normalized to values for the cerebellum. Using values generated from the images, the binding potential of tracers with a K_{D} between 1×10⁻⁹ M-10×10⁻⁹ M were significantly higher in the hippocampus than the cortex. However, tracers with K_{D} ranges above 1×10⁻⁹ M did not show significant differences between these brain compartments. Further, the results demonstrated a positive correlation between the K_{D} of the tracer and the percent error of the calculated beta amyloid concentration values compared to the input concentrations values used to generate the images (P=0.01). Thus, the simulation demonstrates the value of predicting how affinity will affect imaging metrics and, further, confirms the importance of high affinity binding tracers to accurately quantify beta amyloid using PET.

In addition, a sensitivity analysis of virtual traces directed against different species of beta amyloid in response to amyloid precursor protein (APP) cleavage inhibition was performed. One tracer was directed against free beta amyloid peptide monomer and the other was simulated to bind only to the beta amyloid located on the ends of each oligomer species, regardless of length. This models the condition of ‘inaccessibility’ of tracer for beta amyloid molecules located within a fibril or oligomer assembly. The affinity of both virtual tracers in this case was set at 30×10⁻⁹ M, which is consistent with the affinity of beta amyloid for itself in this model (thus we include in vivo competition between beta amyloid and tracer).

The APP processing inhibitor was simulated to be 40% effective at inhibiting beta amyloid production. The model simulated both beta amyloid monomer reduction and total beta amyloid reduction for a one-year course of therapy. The decrease in monomer concentration displayed dramatic kinetics, dropping rapidly within the first hour after administration of the therapy and rapidly reaching steady state within two hours. The decrease in total beta amyloid and beta amyloid attached to the ends of each oligomer is delayed by the time constant of the reversible polymerization process, which depends upon the ratio of e and f. PET simulations show an observable difference in the free beta amyloid monomer compared to higher order species at 24 hours following therapy. As expected, this indicates that PET imaging of beta amyloid monomer would be more sensitive to APP inhibition than beta amyloid contained within higher order oligomer assemblies.

Computational simulation is a powerful, yet under utilized approach to studying complex biological and physical systems. Although this example is focused on beta amyloid imaging, the concept of simulation that links scanners, ligands and target parameters can be applied to many other biological and disease systems using available data to build the desired models. Further, quantitative methods of molecular imaging may benefit from these techniques, not only for the design of optimal tracers, but to couple quantitative results with biological theories, analyses, and interpretations. It is also envisioned that such models may be useful in the interpretation of imaging results such that clinically relevant data can be extrapolated.

In accordance with certain embodiments of present technique, code or blocks of code may be used to generate a set of simulated sensed data based on at least a time activity model, and an imager model as illustrated in FIG. 10 and as described previously. The various embodiments and aspects already described may comprise an ordered listing of executable instructions for implementing logical functions. The ordered listing can be embodied in any computer-readable medium for use by or in connection with a computer-based system that can retrieve the instructions and execute them. In the context of this application, the computer-readable medium can be any means that can contain, store, communicate, propagate, transmit or transport the instructions. The computer readable medium can be an electronic, a magnetic, an optical, an electromagnetic, or an infrared system, apparatus, or device. An illustrative, but non-exhaustive list of computer-readable mediums can include an electrical connection (electronic) having one or more wires, a portable computer diskette (magnetic), a random access memory (RAM) (magnetic), a read-only memory (ROM) (magnetic), an erasable programmable read-only memory (EPROM or flash memory) (magnetic), an optical fiber (optical), and a portable compact disc read-only memory (CDROM) (optical). Note that the computer readable medium may comprise paper or another suitable medium upon which the instructions are printed by mechanical and electronic means or be hand-written. For instance, the instructions can be electronically captured via optical scanning of the paper or other medium, then compiled, interpreted or otherwise processed in a suitable manner if necessary, and then stored in a computer readable memory.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An imaging simulator system, comprising:
a processor assembly comprising:
a time activity module configured to generate time activity data;
an imager module configured to receive at least one imager parameter and to generate an imager model; and
a simulator module configured to receive the imager model and the time activity data and to generate simulated sensed data.

2. The imaging simulator system of claim 1, wherein the time activity module generates the time activity data based on at least one of a biological module and a pharmacokinetic module.

3. The imaging simulator system of claim 1, wherein the time activity module generates the time activity data based on at least one of a phantom module and a pharmacokinetic module.

4. The imaging simulator system of claim 1, wherein the time activity module generates the time activity data via a customizable time activity generator module configurable to generate time activity data based on one or more user provided parameters.

5. The imaging simulator system of claim 1, wherein the time activity data comprises a phantom generated by applying a biological model and a pharmacokinetic model to a phantom model.

6. The imaging simulator system, as recited in claim 1, wherein the biological model comprises at least one of a biochemical model and a biophysical model.

7. The imaging simulator system, as recited in claim 1, wherein the imager model comprises a diagnostic medical imager model.

8. The imaging simulator system, as recited in claim 1, wherein the imager model comprises at least one of a positron emission tomography imaging system model, a SPECT imaging system model, a planar imaging system model, a magnetic resonance scanner model, computed tomography imaging system model, x-ray imaging system model, ultrasound imaging system model, and optical imaging system model.

9. The imaging simulator system, as recited in claim 1, comprising an image reconstruction module configured to receive one or more reconstruction parameters and the simulated sensed data and to generate one or more simulated images.

10. The imaging simulator system, as recited in claim 9, comprising a display device configured to display the one or more simulated images.

11. The imaging simulator system, as recited in claim 9, comprising an image analysis module configured to receive the one or more simulated images and to generate one or more image quality metrics.

12. The imaging simulator system, as recited in claim 11, comprising an image acquisition protocol adjustment module configured to receive the one or more image quality metrics and to generate feedback data for at least one of the time activity module, the imager module, and the image reconstruction module.

13. The imaging simulator system, as recited in claim 1, wherein the pharmacokinetic module is configured to generate the pharmacokinetic model based on at least one of the pharmacokinetic parameters and the biological model.

14. A method of simulating an imaging process, the method comprising the steps of:
generating a set of simulated sensed data based on an imager model and time activity data.

15. The method of claim 14, comprising:
generating the time activity data based on at least a biological module and a pharmacokinetic module.

16. The method of claim 14, comprising:
generating the time activity data based on at least a phantom module and a pharmacokinetic module.

17. The method of claim 14, comprising:
generating the time activity data based on one or more user provided parameters.

18. The method of claim 14, wherein the time activity data comprises a phantom generated by applying a biological model and a pharmacokinetic model to a phantom model.

19. The method, as recited in claim 14, wherein the time activity data is generated by at least one of a biological model, a pharmacokinetic model, a phantom model, and a customizable time activity generator.

20. The method, as recited in claim 19 wherein the biological model comprises at least one of a biochemical model and a biophysical model.

21. The method, as recited in claim 14, wherein generating the set of simulated sensed data comprises integrating the time activity data with the pharmacokinetic model to generate a set of simulated pharmacokinetic data representing pharmacokinetic activity over time.

22. The method, as recited in claim 14, wherein generating the set of simulated sensed data comprises simulating at least one imaging process by processing a set of simulated pharmacokinetic data based on the imager model, wherein the simulated pharmacokinetic data is derived from the time activity data and the pharmacokinetic model.

23. The method, as recited in claim 14, wherein the imager model comprises a diagnostic medical imager model.

24. The method, as recited in claim 14, comprising reconstructing the simulated sensed data to generate one or more simulated images.

25. The method, as recited in claim 24, comprising displaying the one or more simulated images.

26. The method, as recited in claim 24, comprising generating one or more image quality metrics based on the one or more simulated images.

27. The method, as recited in claim 26, comprising generating feedback data based on the one or more image quality metrics.

28. A tangible, machine-readable media, comprising:
code adapted to generate simulated sensed data based on an imager model and time activity data.

29. The tangible, machine-readable media, as recited in claim 28, comprising:
code adapted to generate the time activity data based on at least a biological module and a pharmacokinetic module.

30. The tangible, machine-readable media, as recited in claim 28, comprising:
code adapted to generate the time activity data based on at least a phantom module and a pharmacokinetic module.

31. The tangible, machine-readable media, as recited in claim 28, comprising:
code adapted to generate the time activity data based on one or more user provided parameters.

32. The tangible, machine-readable media, as recited in claim 28, wherein the time activity data comprises a phantom generated by applying a biological model and a pharmacokinetic model to a phantom model.

33. The tangible, machine-readable media, as recited in claim 28, wherein the time activity data is generated by at least one of a biological model, a pharmacokinetic model, a phantom model, and a customizable time activity generator.

34. The tangible, machine-readable media, as recited in claim 28, comprising code adapted to reconstruct the simulated sensed data to generate one or more simulated images.

35. The tangible, machine-readable media, as recited in claim 34, comprising code adapted to display the one or more simulated images.

36. The tangible, machine-readable media, as recited in claim 34, comprising code adapted to generate one or more image quality metrics based on the one or more simulated images.

37. The tangible, machine-readable media, as recited in claim 36, comprising code adapted to generate feedback data based on the one or more image quality metrics.

38. An imaging simulator system, comprising:
means for generating a set of simulated sensed data based on an imager model and time activity data;

39. A method for selecting an imaging compound, the method comprising the steps of:
providing at least one of a time activity data and an imager model to a simulation system;
obtaining a set of simulation results from the simulation system, wherein the set of simulation results are generated by the simulation system based on the imager model and the time activity data; and
selecting an imaging compound for use or development based upon the set of simulation results.

40. The method, as recited in claim 39, wherein the time activity data is generated by at least one of a biological module, a pharmacokinertic module, a phantom module, and a customizable time activity generator.

41. The method, as recited in claim 40, wherein the biological module comprises at least one a biochemical model and a biophysical model.

42. The method, as recited in claim 39, wherein the set of simulation results comprises one or more simulated images.
